(19) United States
(12) Patent Application Publication
(10) Pub. No.: US 2003/0144341 A1
(43) Pub. Date: Jul. 31, 2003

(54) Cholesterol-lowering agents as treatment for psychological and cognitive disorders

(76) Inventors: Perry F. Renshaw, Arlington, MA (US); Bruce M. Cohen, Lexington, MA (US)

Correspondence Address: CLARK & ELBING LLP, 101 FEDERAL STREET, BOSTON, MA, 02110, US

(21) Appl. No.: 10/375,435

(22) Filed: Feb. 27, 2003

Related U.S. Application Data
(63) Continuation of application No. 10/071,706, filed on Feb. 7, 2002, now abandoned.
(60) Provisional application No. 60/267,333, filed on Feb. 7, 2001.

(51) Int. Cl. A61K 31/401; A61K 31/366; A61K 31/225

(52) U.S. Cl. 514/423; 514/460; 514/548

(57) ABSTRACT

Provided are methods for treating a membrane fluidity-related cognitive or psychological disorder in a human patient. The methods include the steps of: (a) performing a diagnostic test on the patient to determine that the patient has a cognitive or psychological disorder, and (b) administering to the patient a cholesterol-lowering agent in an amount sufficient to lower the serum cholesterol of the patient enough to increase brain membrane fluidity adequately to treat the cognitive or psychological disorder.

### CROSS REFERENCE TO RELATED APPLICATIONS

This application claims benefit of U.S. provisional application serial No. 60/267,333 filed Feb. 7, 2001.

### FIELD OF THE INVENTION

The invention relates to methods for treating membrane fluidity-related psychological and cognitive disorders.

### BACKGROUND

Cell membranes define the boundaries of cells and perform a variety of important cellular functions. One of their primary functions is to control the traffic of substances into and out of the cell. They also play a vital role in cell-cell recognition, adhesion, communication, and signaling.

The composition of a cell membrane determines its microscopic structure, which in turn, affects such parameters as membrane shape, permeability, and fluidity, as well as the conformation and functionality of ion channels, enzymes, and receptors that are embedded within the membrane. Lipids and proteins are the primary components of cell membranes, although carbohydrates may also be present. Phospholipids, such as phosphatidylcholine and sphingomyelin, are the most abundant membrane lipids. Glycolipids and cholesterol are also prevalent.

Alterations in membrane lipid order and composition can have a profound impact on the physical and chemical properties of the membrane. For instance, changes in the membrane's cholesterol-to-phospholipid ratio can lead to changes in membrane fluidity. Generally, an increase in cholesterol content results in a decrease in membrane fluidity, while a reduction in membrane cholesterol tends to increase fluidity. Even relatively small changes in membrane fluidity can induce considerable effects on membrane-linked functions, including ion transport, signal recognition and transduction, and the regulation of enzyme activities.

### SUMMARY OF THE INVENTION

We posit that changes in the ratio of cholesterol to other cell membrane components can have a significant impact on brain health and functioning, and that many of the adverse symptoms associated with certain cognitive and psychological disorders are the result of elevated levels of cholesterol in brain cell membranes. We believe that as membrane cholesterol levels increase, the resulting decrease in neuronal membrane fluidity interferes with the proper functioning of brain cells. A reduction in the amount of cholesterol incorporated into brain cell membranes, which results in an increase in membrane fluidity, can be accomplished by reducing serum cholesterol levels.

Accordingly, the present invention features a method for treating a membrane fluidity-related cognitive or psychological disorder in a human patient, which method includes the steps of: (a) performing a diagnostic test on the patient to determine that the patient has a cognitive or psychological disorder and, if the patient has been so diagnosed, (b) administering to the patient a cholesterol-lowering agent in an amount sufficient to lower the serum cholesterol of the patient enough to increase brain membrane fluidity adequately to treat the cognitive disorder.

The cholesterol-lowering agent can be selected from the group consisting of clofibrate, colestipol, gemfibrozil, lovastatin, cerivastatin, fluvastatin, atorvastatin, pravastatin, and simvastatin. Preferably, the agent is a statin, such as atorvastatin or simvastatin.

The method of the invention may be used to treat a variety of membrane fluidity-related disorders, including cognitive and affective disorders, such as depression, dysthymia, cyclothymia, bipolar disorder, schizoaffective disorder, age-related memory loss, mild cognitive impairment, and dementia. In addition, the method is useful in the treatment of substance abuse disorders, including alcohol, stimulant, opiate, marijuana, solvent, and nicotine abuse or dependence

Other features and advantages of the invention will be apparent from the following detailed description thereof and from the claims.

### DEFINITIONS

By “cholesterol-lowering agent” is meant a chemical compound or composition capable of lowering the serum cholesterol level of a human.

By “affective disorder” is meant any emotional or mental disorder characterized primarily by disturbances in mood.

By “cognitive disorder” is meant any disorder that affects mental processes, including impairments of memory, learning, awareness, attention, communication, intellectual capacity, judgement-making ability, and/or motor coordination. Such disorders are often accompanied by personality and behavioral changes. Examples of these disorders include, but are not limited to delirium, dementia, and amnestic disorders.

By “substance abuse disorder” is meant any physiological or psychological disorder characterized primarily by the abuse of, addiction to, or dependence on a chemical substance.

By “membrane fluidity-related” is meant associated with a change (either a decrease or increase) in cell membrane fluidity or membrane order.

### DETAILED DESCRIPTION

The present invention provides a method of treating mental disorders that are associated with a decrease in brain cell membrane fluidity. The method involves administering to a patient a cholesterol-lowering agent that is capable of reducing the patient's level of serum cholesterol. When serum cholesterol levels are decreased, there is a corresponding decrease in the level of cholesterol in brain cell membranes. This reduction leads to an increase in membrane fluidity. Thus, by lowering serum cholesterol levels through the administration of a cholesterol-lowering agent, it is possible to increase membrane fluidity, thereby reducing or eliminating the adverse effects associated with certain mental disorders.

Diagnosis of Psychological and Cognitive Disorders

The initial step of this method involves diagnosing a human patient to determine whether the individual is suffering from a condition that is associated with a membrane fluidity abnormality. A number of psychological and cognitive disorders are marked by changes in the lipid composition of brain cell membranes, which result in a decrease in membrane fluidity. This reduction in membrane fluidity can lead to a variety of mental impairments, which may be treated by restoring proper fluidity though the administration of cholesterol-lowering agents. Examples of psychological disorders which may be characterized by a decrease in membrane fluidity include, but are not limited to, affective disorders, such as major depression, dysthymia, cyclothymia, bipolar disorder, schizoaffective disorder, and borderline personality disorder.

Cognitive disorders are often age-related or the result of neurodegenerative disease processes, and can also be accompanied by changes in neuronal membrane lipid composition and fluidity. Some of the adverse symptoms of these disorders may stem from a decrease in brain cell membrane fluidity, and can therefore be treated by administration of cholesterol-lowering agents. Exemplary membrane fluidity-related cognitive disorders include, but are not limited to, age-related memory loss, mild cognitive impairment, and dementia of any etiology (e.g., Alzheimer Disease, Parkinson's Disease, Creutzfeldt-Jakob Disease, Huntington's Disease, Pick's Disease, HIV, head trauma, etc.)

Overuse of certain psychoactive substances may also effect brain cell membrane composition and fluidity. The method of the invention may, therefore, be useful in the treatment of various substance abuse disorders, including but not limited to, alcohol, stimulant (e.g., cocaine and methamphetamine), opiate, marijuana, solvent, and nicotine abuse and dependence.

Cognitive and psychological disorders, including affective disorders and substance abuse disorders, can be diagnosed using a variety of well-known testing procedures. Two commonly used systems for diagnosing such disorders are the Diagnostic and Statistical Manual of Mental Disorders (DSM-IV) and the International Classification of Disease (ICD-10). These systems provide a set of standard criteria for effectively and reliably diagnosing a broad range of mental disorders. In some circumstances, biochemical and serological methods may also be available for diagnosing these disorders, and may be used alone or in conjunction with other diagnostic methods, including psychological testing. Of course, the method of diagnosis will vary depending on the condition of the patient and the nature of the disorder being diagnosed.

Administration of Cholesterol-Lowering Agents

Once a patient has been diagnosed with a membrane fluidity-related cognitive or psychological disorder, the patient is treated by administration of cholesterol-lowering agents in order to restore proper brain cell membrane fluidity, thereby alleviating the symptoms associated with decreased fluidity. A wide variety of cholesterol-lowering agents are known in the art and may be used in the present invention. Examples of suitable cholesterol-lower agents include, but are not limited to, clofibrate (ATROMID-S®), colestipol (COLESTID®), gemfibrozil (LOPID® or GEMCOR®), and various statins, such as fluvastatin (LESCOL®), atorvastatin (LIPITOR®), pravastatin (PRAVACHOL®), lovastatin. (MEVACOR®), cerivastatin (BAYCOL®), and simvastatin (ZOCOR®). Methods for preparing these and other cholesterol-lowering agents are well known in the art and many are commercially available medications.

The cholesterol-lowering agents can be administered systemically, e.g. orally or by IM or IV injection, in admixture with a pharmaceutically acceptable carrier adapted for the route of administration. A variety of physiologically acceptable carriers can be used to administer the cholesterol-lowering agents and their formulations are known to those skilled in the art and are described, for example, in Remington's Pharmaceutical Sciences, (18^{th} edition), ed. A. Gennaro, 1990, Mack Publishing Company, Easton, Pa. and Pollock et al.

Oral ingestion is the preferred route of administration. Compositions intended for oral use can be prepared in solid or liquid forms, according to any method known to the art for the manufacture of pharmaceutical compositions. The compositions may optionally contain sweetening, flavoring, coloring, perfuming, and preserving agents in order to provide a more palatable preparation.

Solid dosage forms for oral administration include capsules, tablets, pills, powders, and granules. Generally, these pharmaceutical preparations contain active ingredient admixed with non-toxic pharmaceutically acceptable excipients. These may include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium, and can also include adjuvants, such as wetting agents, emulsifying agents, and suspending agents.

Alternatively, the pharmaceutical compositions can be administered parenterally (e.g., by intramuscular, intraperitoneal, intravenous, or subcutaneous injection or implant). Formulations for parenteral administration include sterile aqueous or non-aqueous solutions, suspensions, or emulsions. A variety of aqueous carriers can be used, e.g., water, buffered water, 0.4 percent saline, and the like. Examples of other suitable vehicles include polypropylene glycol, polyethylene glycol, vegetable oils, gelatin, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

The cholesterol-lowering agents can also be administered in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

Dosage

The amount of active ingredient that is combined with the carrier materials to produce a single dosage will vary depending upon the subject being treated and the particular mode of administration. Generally, the cholesterol-lowering agent should be administered in an amount sufficient to lower the serum cholesterol level of the patient. The level should be lowered sufficiently to increase the patient's brain cell membrane fluidity (i.e., the amount administered should be sufficient to cure or at least partially arrest the symptoms of the disorder and its complications). Membrane fluidity can be monitored using T2 MR mapping, a technique that indirectly measures the physical properties of the outer leaflet of the lipid bilayer of cell membranes and is described in U.S. Ser. No. 60/254,279, which is hereby incorporated by reference. T2 mapping works by measuring the relative movement of water molecules in the immediate vicinity of the cell membrane. A decrease in the amount of cholesterol incorporated into the cell membrane, which would result in an increase in fluidity, would be observed as a change in the T2 signal.

Dosage levels on the order of about 0.1 mg to about 400 mg per kilogram of body weight per day (from about 1.0 mg to about 30.0 g per 70 kg patient per day) are useful in the treatment of the above mentioned psychological and cognitive disorders. The daily dosage may be administered as a single dose or divided into multiple doses. Typically, patients take one or two capsules orally, three to four times per day (e.g., once in the morning, once in the early afternoon, and again in the evening). In general, the desired daily dosage should be taken for a prolonged period, usually at least two weeks, preferably four to six weeks, although longer periods of administration of two months or more may be needed.

Suitable dosage ranges for several well-known cholesterol-lowering agents are provided in the following table.

Table 1. Dosage ranges for commonly used cholesterol-lowering agents.

| Cholesterol-Lowering | Daily Dosage Range |
|---|---|
| Agent | (per 70 kg of body weight) |
| Clofibrate | 500 to 2000 mg |
| Colestipol | 5 to 30 g |
| Gemfibrozil | 1200 mg |
| Fluvastatin | 20 to 40 mg |
| Atorvastatin | 20 to 80 mg |
| Simvastatin | 5 to 80 mg |
| Pravastatin | 10 to 20 mg |
| Lovastatin | 20 to 80 mg |
| Cerivastatin | 0.2 to 0.8 mg |

One skilled in the art will appreciate that the exact individual dosages may be adjusted somewhat depending on a variety of factors, including the specific cholesterol-lowering agent being administered, the time of administration, the route of administration, the nature of the formulation, the rate of excretion, the particular disorder being treated, the severity of the disorder, and the age, weight, health, and gender of the patient. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well-known in the art. The precise therapeutically effective dosage levels and patterns are preferably determined by the attending physician in consideration of the above identified factors.

With respect to substance abuse disorders, the dose level for suppressing an urge to consume the abused substance may vary among individuals depending upon the severity of the individual's symptoms and/or the individual's predisposition or susceptibility to substance abuse. The optimum dosage can generally be determined by monitoring the amount of substance used by the individual while on the medication or by the intensity of the individual's desire for the abused substance.

In addition to treating pre-existing cognitive, affective, and substance abuse disorders, cholesterol-lowering agents can be administered prophylactically in order to prevent or slow the onset of these disorders. In prophylactic applications, a pharmaceutical composition containing a cholesterol-lowering agent is administered to a patient susceptible to or otherwise at risk of a particular membrane fluidity-related disorder. Again, the precise amounts that are administered depend on various factors such as the patient's state of health and weight, but generally range from about 0.5 mg to about 5,000 mg per 70 kilogram patient, more commonly from about 5 mg to about 2,000 mg per 70 kg of body weight.

Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein.

All publications and patents mentioned in this specification are hereby incorporated by reference.

What is claimed is:

1. A method of treating a membrane fluidity-related cognitive disorder in a human patient, said method comprising the steps of:
a) performing a diagnostic test on said patient to determine that said patient has a cognitive disorder, and
b) administering a cholesterol-lowering agent to said patient in an amount sufficient to lower the serum cholesterol of said patient sufficiently to increase brain membrane fluidity sufficiently to treat said cognitive disorder.

2. The method of claim 1, wherein said cognitive disorder is selected from the group consisting of age-related memory loss, mild cognitive impairment, and dementia.

3. The method of claim 2, wherein said cognitive disorder is age-related memory loss.

4. The method of claim 2, wherein said cognitive disorder is dementia.

5. The method of claim 4, wherein said dementia is associated with Alzheimer's disease.

6. The method of claim 1, wherein said cholesterol-lowering agent is selected from the group consisting of clofibrate, colestipol, gemfibrozil, fluvastatin, atorvastatin, pravastatin, lovastatin, cerivastatin, and simvastatin.

7. The method of claim 6, wherein said cholesterol-lowering agent is a statin.

8. The method of claim 7, wherein said statin is simvastatin.

9. The method of claim 7, wherein said statin is pravastatin.

10. The method of claim 7, wherein said statin is atorvastatin.

11. The method of claim 6, wherein said cholesterol-lowering agent is administered orally.

12. A method of treating a membrane fluidity-related affective disorder in a human patient, said method comprising the steps of:
a) performing a diagnostic test to determine that the patient has a membrane fluidity-related affective disorder, and
b) administering a cholesterol-lowering agent to said patient in an amount sufficient to lower the serum cholesterol of said patient sufficiently to increase brain membrane fluidity sufficiently to treat said affective disorder.

13. The method of claim 12, wherein said affective disorder is selected from the group consisting of depression, dysthymia, cyclothymia, bipolar disorder, schizoaffective disorder, and borderline personality disorder.

14. The method of claim 13, wherein said affective disorder is depression.

15. The method of claim 13, wherein said affective disorder is schizoaffective disorder.

16. The method of claim 13, wherein said affective disorder is bipolar disorder.

17. The method of claim 12, wherein said cholesterol-lowering agent is selected from the group consisting of clofibrate, colestipol, gemfibrozil, fluvastatin, atorvastatin, pravastatin, lovastatin, cerivastatin, and simvastatin.

18. The method of claim 17, wherein said cholesterol-lowering agent is a statin.

19. The method of claim 18, wherein said statin is simvastatin.

20. The method of claim 18, wherein said statin is pravastatin.

21. The method of claim 18, wherein said statin is atorvastatin.

22. The method of claim 17, wherein said cholesterol-lowering agent is administered orally.

23. A method of treating a membrane fluidity-related substance abuse disorder in a human patient, said method comprising the steps of:
a) performing a diagnostic test to determine that the patient has a membrane fluidity-related substance abuse disorder, and
b) administering a cholesterol-lowering agent to said patient in an amount sufficient to lower the serum cholesterol of said patient sufficiently to increase brain membrane fluidity sufficiently to treat said substance abuse disorder.

24. The method of claim 23, wherein said substance abuse disorder is characterized by an abuse of or dependence on a substance selected from the group consisting of alcohol, stimulants, opiates, marijuana, solvents, and nicotine.

25. The method of claim 24, wherein said substance is alcohol.

26. The method of claim 24, wherein said substance is cocaine.

27. The method of claim 24, wherein said substance is methamphetamine.

28. The method of claim 23, wherein said cholesterol-lowering agent is selected from the group consisting of clofibrate, colestipol, gemfibrozil, fluvastatin, atorvastatin, pravastatin, lovastatin, cerivastatin, and simvastatin.

29. The method of claim 28, wherein said cholesterol-lowering agent is a statin.

30. The method of claim 29, wherein said statin is simvastatin.

31. The method of claim 29, wherein said statin is pravastatin.

32. The method of claim 29, wherein said statin is atorvastatin.

33. The method of claim 28, wherein said cholesterol-lowering agent is administered orally.
